(19) United States
(12) Patent Application Publication
(10) Pub. No.: US 2019/0091316 A1
(43) Pub. Date: Mar. 28, 2019

(54) Subcutaneous Delivery of Adenovirus with Dual Targeting

(71) Applicants: Nant Holdings IP, LLC, Culver City, CA (US); NantCell, Inc., Culver City, CA (US)

(72) Inventors: Patrick Soon-Shiong, Culver City, CA (US); Kayvan Niazi, Culver City, CA (US); Shahrooz Rabizadeh, Culver City, CA (US)

(21) Appl. No.: 16/075,874

(22) PCT Filed: Feb. 12, 2017
(86) PCT No.: PCT/US2017/017588

§ 371 Date: Aug. 6, 2018

Related U.S. Application Data
(60) Provisional application No. 62/294,251, filed on Feb. 11, 2016.
(60) Provisional application No. 62/294,987, filed on Feb. 12, 2016.

(51) Int. Cl. A61K 39/00; A61K 9/00; C12N 7/00; C07K 14/74; C07K 14/705; C07K 16/28; A61K 35/17

(52) U.S. Cl. CPC A61K 39/001114; A61K 9/0019; C12N 7/00; C07K 14/70539; C07K 14/70532; C12N 2710/10034; A61K 35/17; C07K 2319/02; C07K 2319/06; C07K 2319/30; C12N 2710/10043; C07K 16/2818

(57) ABSTRACT

Immunotherapeutic methods and compositions are contemplated in which neoepitopes and/or tumor associated antigens are delivered to dendritic cells via an adenoviral expression system that targets MHC-I and/or MHC-II presentation systems and that further provides one or more recombinant peptides to stimulate T cell activation and interfere with checkpoint inhibition. Treatment is further supported by transfusion of NK cells, which may be modified to have a high affinity CD 16 receptor and/or a chimeric antigen receptor that binds to one or more neoepitopes and/or tumor associated antigens.

This application claims priority to U.S. Provisional Application Ser. No. 62/294251, filed Feb. 11, 2016, and further claims priority to U.S. Provisional Application Ser. No. 62/294987, filed Feb. 12, 2016, both of which are incorporated herein by reference.

### FIELD OF THE INVENTION

The field of the invention is immunotherapeutic compositions and methods, especially as it relates to cancer vaccine preparations that target the MHC-I and/or MHC-II presentation pathways, particularly with concurrent modulation of checkpoint inhibition.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cancer vaccines have shown much promise, but are often limited in practice due to various factors, including immunogenicity of the viral vehicle and/or poor presentation of the recombinant antigen. Notably, poor presentation may not only arise from the antigen per se but also from a poor match to a patient's particular HLA type. Furthermore, and especially where recombinant viruses are used to produce a therapeutic antigen, systemic delivery and low infectivity together with the patient's clearance of the virus tends to prevent effective and pervasive training of the various components of the patient's immune system (e.g., dendritic cells, CD8+ T cells, CD4+ helper T cells, B-cells) is often not or only poorly achieved. In addition, even if antigen presentation is achieved to at least some degree, various regulatory mechanisms, and especially immune checkpoint inhibition, often present an additional hurdle to effective treatment.

For example, U.S. Pat. No. 7,118,738 teaches use of a poxvirus that carries recombinant DNA encoding MUC1 as a cancer associated antigen and reports that an immune reaction can be augmented using B7.1 and/or B7.2 as adjuvants. However, such viruses have not proven to consistently elicit a therapeutically effective immune response. Similarly, CEA/TRICOM was expressed from a recombinant poxvirus (see e.g., Clin Cancer Res 2005, Vol. 11, 2416-2426). However, immune stimulation from TRICOM was less than desired. Moreover, CEA was also expressed on cells other than cancer cells, and the poxvirus as a delivery system has been shown to be immunogenic after first administration. Additionally, immune stimulation in such system was not strongest against CEA, but rather precipitated an immune response against other proteins in an antigen cascade. Thus, while stimulatory adjuvants hold at least conceptually promise, their practical success was often limited.

In still further known methods, a viral vector for expression of an antigen (e.g., CEA, MUC1, brachyury) was co-administered with a checkpoint inhibitor to enhance an immune response as described in WO 2016/172249, US 2016/0101170, and US 2016/0339090. Use of checkpoint inhibitors has shown in at least some cancers remarkable success. However, due to the typically systemic administration of checkpoint inhibitors, undesirable side effects are often a significant risk.

Regardless of the particular delivery, it should be appreciated that the generation of a durable immune response requires not only proper antigen processing and presentation, but also proper formation of an immune synapse and propagation of the antigen stimulus through various components of the immune system to so produce a therapeutically effective humoral and cellular response. Currently known systems and methods generally fail to provide such coordinated activities.

Therefore, even though numerous methods and compositions to generate an immune response are known in the art, all or almost all of them suffer from various disadvantages. Thus, there remains a need for improved compositions and methods for immunotherapy, and especially for cancer immune therapy.

### SUMMARY OF THE INVENTION

The inventive subject matter is directed to compositions and methods of generating an immune response addressing the above issues by subcutaneous administration of recombinant and preferably non-immunogenic viruses that infect antigen presenting cells (e.g., dendritic cells) to drive production, processing, and presentation of cancer-related epitopes wherein the epitopes are specifically directed towards MHC-I and MHC-II presentation pathways to improve antigen presentation. Moreover, to achieve an even more robust immune response, infected cells will further express various co-stimulatory molecules as well as peptides that interfere with checkpoint receptors of immune competent cells (and especially on T cells and NK-cells). Where desired, checkpoint inhibitors may also be subcutaneously injected at or near the site of administration of the recombinant virus and may as such not be encoded in the viral recombinant nucleic acid. Because of the targeted antigen presentation using MHC-I and MHC-II presentation pathways, an immune response is propagated via CD8+ and CD4+ T cells, respectively, that are material to instructing NK and B-cells as well as the generation of cytotoxic T cells. An immune response may still further be augmented by subsequent or later administration of NK cells, and most preferably genetically engineered NK cells as is further described in more detail below.

In one aspect of the inventive subject matter, the inventors contemplate a method of treating a patient having a tumor. Especially contemplated methods will include a step of subcutaneously administering a recombinant virus comprising a nucleic acid that encodes (a) at least one tumor-related epitope of the tumor of the patient; (b) at least one co-stimulatory molecule; and (c) a peptide that binds to a checkpoint receptor. Most typically, the nucleic acid further includes a trafficking signal to direct a peptide product encoded by the nucleic acid to the cytoplasm, the endosomal compartment, and/or the lysosomal compartment. In yet another step, NK cells are administered to the patient.

Preferably, but not necessarily, the recombinant virus is an adenovirus, optionally with a deleted or non-functional E2b gene to reduce immunogenicity. It is still further contemplated that the tumor-related epitope is an HLA-matched tumor-related epitope, which may be a cancer associated epitope, a cancer-specific epitope, or a patient- and tumor-specific neoepitope.

With respect to the co-stimulatory molecule it is contemplated that the co-stimulatory molecule is B7.1 (CD80), B7.2 (CD86), ICAM-1 (CD54), ICOS-L, LFA-3 (CD58), 4-1BBL, CD30L, CD40, CD40L, CD48, CD70, CD112, CD155, GITRL, OX40L, or TL1A, and preferred peptides that binds to the checkpoint receptor will bind to CTLA-4 (CD152) and/or PD-1 (CD 279).

In further contemplated methods, the trafficking signal directs the peptide product to the cytoplasm, to the endosomal compartment, and/or to the lysosomal compartment. Therefore, suitable trafficking signals include cytoplasmic retention sequences, endosomal targeting sequences, and/or lysosomal targeting sequences. For example, the nucleic acid may have a first trafficking signal that directs a first peptide product to the cytoplasm and a second trafficking signal that directs a second peptide product to the endosomal or lysosomal compartment, with first and second peptide products being identical or distinct. Additionally, it is contemplated that the peptide product(s) may further include a sequence portion that enhances intracellular turnover of the peptide product.

With respect to suitable NK cells it is generally contemplated that the NK cells are genetically modified such that the NK cells (1) have a reduced or abolished expression of at least one killer cell immunoglobulin-like receptor, (2) express a high-affinity Fcγ receptor, (3) express a chimeric T cell receptor, and/or (4) have a deletion in NKG2A. Most typically, the NK cells are administered between one and 14 days after subcutaneously administering the recombinant virus.

In another aspect of the inventive subject matter, the inventors contemplate a method of stimulating a CD8+ T cell response in a patient having a tumor that typically includes a step of subcutaneously administering a recombinant virus that comprises a nucleic acid that encodes (a) at least one tumor-related epitope of the tumor of the patient, operably coupled to a trafficking signal that retains the at least one tumor-related epitope in the cytoplasm; (b) a plurality of co-stimulatory molecules, at least one of which is B7.1 (CD80) or B7.2 (CD86); and (c) a peptide that binds to at least one of PD-1 and CTLA-4. In another step, NK cells are administered to the patient.

Alternatively, in yet another aspect of the inventive subject matter, the inventors contemplate a method of stimulating a CD4+ T cell response in a patient having a tumor that comprises a step of subcutaneously administering a recombinant virus that comprises a nucleic acid that encodes (a) at least one tumor-related epitope of the tumor of the patient, operably coupled to a trafficking signal that directs the at least one tumor-related epitope to the cytoplasm or the endosomal or lysosomal compartment; (b) a plurality of co-stimulatory molecules, at least one of which is B7.1 (CD80) or B7.2 (CD86); and (c) a peptide that binds to at least one of PD-1 and CTLA-4. IN still another step, NK cells are administered to the patient.

Most typically, the recombinant virus in such methods is an adenovirus, optionally with a deleted or non-functional E2b gene to reduce immunogenicity. As noted above, suitable tumor-related epitopes will further include a sequence portion that enhances intracellular turnover of the tumor-related epitope. For example, such epitopes may be an HLA-matched cancer associated epitope, an HLA-matched cancer-specific epitope, or an HLA-matched patient- and tumor-specific neoepitope.

In addition, it is contemplated that the plurality of co-stimulatory molecules may further include at least one additional co-stimulatory molecule selected form the group consisting of ICAM-1 (CD54), ICOS-L, LFA-3 (CD58), 4-1BBL, CD30L, CD40, CD40L, CD48, CD70, CD112, CD155, GITRL, OX40L, and TL1A, and/or that the peptide that binds to at least one of PD-1 and CTLA-4 is a membrane bound antibody fragment. Moreover, it is contemplated that the NK cells are genetically modified NK cells that (1) have a reduced or abolished expression of at least one killer cell immunoglobulin-like receptor, (2) express a high-affinity Fcγ receptor, (3) express a chimeric T cell receptor, and/or (4) have a deletion in NKG2A.

Moreover, the inventors also contemplate that all methods presented herein may further include a step of administering a low dose chemotherapy and/or low dose radiation therapy to the patient under a protocol effective to trigger expression or increase expression of a NKG2D ligand on the cells of the tumor. Where desired contemplated methods may further include a step of identifying new neoepitopes in residual tumor cells and modifying the recombinant virus to include at least one of the new neoepitopes.

Therefore, and viewed form a different perspective, the inventors also contemplate a viral vector (e.g., recombinant adenovirus genome, optionally with a deleted or non-functional E2b gene) that comprises a nucleic acid that encodes (a) at least one tumor-related epitope of a tumor of a patient; (b) at least one co-stimulatory molecule; and (c) a peptide that binds to a checkpoint receptor. Most typically, the nucleic acid will further include a trafficking signal to direct a peptide product encoded by the nucleic acid to the cytoplasm, the endosomal compartment, or the lysosomal compartment, and the peptide product will further comprise a sequence portion that enhances intracellular turnover of the peptide product. As noted earlier, the tumor-related epitope is preferably an HLA-matched tumor-related epitope (e.g., a cancer associated epitope, a cancer-specific epitope, or a patient- and tumor-specific neoepitope). Similarly, it is preferred that the co-stimulatory molecule is B7.1 (CD80), B7.2 (CD86), ICAM-1 (CD54), ICOS-L, LFA-3 (CD58), 4-1BBL, CD30L, CD40, CD40L, CD48, CD70, CD112, CD155, GITRL, OX40L, or TL1A, and/or that the peptide that binds to the checkpoint receptor binds to CTLA-4 (CD152) or PD-1 (CD 279), optionally comprising a membrane bound antibody fragment.

Therefore, the inventors also contemplate a recombinant virus comprising the viral vector as described above. Likewise, the inventors also contemplate a pharmaceutical composition that includes a recombinant virus as described herein.

Various objects, features, aspects and advantages of the inventive subject matter will become more apparent from the following detailed description of preferred embodiments.

### DETAILED DESCRIPTION

The inventors have now discovered that cancer immune therapy can be significantly improved by use of a preferably subcutaneously administered recombinant virus and immune modulators in combination with NK cell-based therapy.

More specifically, using contemplated methods and compositions presented herein, it is contemplated that by targeting one or more tumor-related epitopes to one or more MHC presentation pathways an immune response can be propagated through both CD8+ and CD4+ T cell populations, which will in turn help generate humoral and cell-based adaptive immune responses. In addition, contemplated methods also employ (preferably genetically modified) NK cells to augment an innate immune response as described in more detail below. Where subcutaneously administered, it is contemplated that viral delivery is particularly effective in infecting dendritic cells, and that subcutaneous administration of checkpoint inhibitors at or near the site of viral injection (e.g., via expression from virally infected cells or via injection) will further augment an immune response at substantially reduced risk for positive cytokine feedback loops (i.e., cytokine storm).

Co-expression/coordinated presence of antigens, co-stimulatory molecules, and checkpoint inhibitors is thought to promote formation of an immune synapse for a duration that is sufficient for activation of T cells, and especially CD8+ and CD4+ T cells. In especially preferred aspects, presence of these entities is ensured by co-expression of the antigens and co-stimulatory molecules from a virus that infects antigen presenting cells, and especially dendritic cells, which may also co-express one or more molecules that bind to CTLA-4 and/or PD-1 as is further discussed in more detail below. Alternatively, or additionally, one or more checkpoint inhibitors (ipilimumab, nivolimumab, etc.) may be injected at or near the site of virus delivery. Most typically, such delivery will be via subcutaneous or subdermal injection. To further enhance antigen processing and presentation, it is contemplated that the expressed antigens will include trafficking sequences that purposely direct the expressed protein to a desired compartment (e.g., cytosolic compartment for MHC-I presentation, or endosomal or lysosomal compartment for MHC-II presentation). Moreover, to enhance antigen processing, it is generally preferred to include one or more ubiquitination sites or include an uncleavable ubiquitin.

As such immune therapy is thought to trigger a strong adaptive immune response with respect to the specific expressed antigen(s), it is further expected that the immune response will also contribute to antigen cascading and additional immune response to newly presented antigens. To even further complement the adaptive immune response, it is generally preferred that components of the innate immune response may be administered to the patient, and especially genetically modified NK cells. For example, and as further discussed in more detail below, NK cells may be genetically modified NK92 cells with a high affinity variant of CD16 to enhance humoral response and/or genetically modified NK92 cells with a chimeric antigen receptor that has a binding domain that is specific to one or more of the tumor related antigens.

With respect to contemplated tumor related epitopes it should be appreciated that any epitope that is associated with a cancer, specific to a particular type of cancer, or that is specific to a patient and tumor (neoepitope) is suitable for use herein, particularly where the epitope is expressed (preferably above expression level of healthy tissue of the same patient) and has a desirable affinity for the patients HLA system. In this context, the term tumor related epitope includes short peptides (e.g., 8-30 amino acids), as well as protein fragments, and even entire proteins.

For example, there are numerous antigens with known association with cancer, and all of those are deemed suitable for use herein, including CEA, MUC-1, EphA3, and CYPB1, and portions thereof. Similarly, there are numerous cancer specific antigens known in the art, such as Her-2, PSA, brachyury, etc., and all of them and portions thereof are deemed suitable for use herein. However, in particularly preferred aspects, tumor related epitopes will include patient and tumor specific neoepitopes. Thus, it should be appreciated that a recombinant virus or viral nucleic acid construct (or nucleic acid construct for expression in a host cell) will include a recombinant segment that encodes at least one (e.g., at least two, three, four, etc.) tumor related epitopes plus at least one co-stimulatory molecule and preferably (but not necessarily) a protein that interferes with checkpoint signaling. Of course, it should be appreciated that where the length of sequences for tumor related epitopes, co-stimulatory molecules, and other proteins exceeds the viral capacity for recombinant nucleic acids, multiple and distinct recombinant viruses may be used.

Sequence information for contemplated tumor related epitopes can be obtained from various publicly known sources (e.g., TCGA, COSMIC, etc.) or can be obtained from the patient, for example, using biopsy samples following standard tissue processing protocol and sequencing protocols. While not limiting to the inventive subject matter, it is typically preferred that the sequence data are patient matched tumor data for patient and tumor-specific neoepitopes (e.g., tumor versus same patient normal), and that the data format is in SAM, BAM, GAR, or VCF format. However, non-matched or matched versus other reference (e.g., prior same patient normal or prior same patient tumor, or homo statisticus) are also deemed suitable for use herein. Therefore, the omics data may be ‘fresh’ omics data or omics data that were obtained from a prior procedure (or even different patient).

Neoepitopes can be characterized as expressed random mutations in tumor cells that created unique and tumor specific antigens. Therefore, viewed from a different perspective, neoepitopes may be identified by considering the type (e.g., deletion, insertion, transversion, transition, translocation) and impact of the mutation (e.g., non-sense, missense, frame shift, etc.), which may as such serve as a first content filter through which silent and other non-relevant (e.g., non-expressed) mutations are eliminated. It should further be appreciated that neoepitope sequences can be defined as sequence stretches with relatively short length (e.g., 7-11 mers) wherein such stretches will include the change(s) in the amino acid sequences. Most typically, the changed amino acid will be at or near the central amino acid position. For example, a typical neoepitope may have the structure of A₄-N-A₄, or A₃-N-A₅, or A₂-N-A₇, or A₅-N-A₃, or A₇-N-A₂, where A is a proteinogenic amino acid and N is a changed amino acid (relative to wild type or relative to matched normal). However, the changed amino acid may also be located at the termini of the neoepitope sequence. For example, neoepitope sequences as contemplated herein include sequence stretches with relatively short length (e.g., 5-30 mers, more typically 7-11 mers, or 12-25 mers) wherein such stretches include the change(s) in the amino acid sequences.

Thus, it should be appreciated that a single amino acid change may be presented in numerous neoepitope sequences that include the changed amino acid, depending on the position of the changed amino acid. Advantageously, such sequence variability allows for multiple choices of neoepitopes and so increases the number of potentially useful targets that can then be selected on the basis of one or more desirable traits (e.g., highest affinity to a patient HLA-type, highest structural stability, etc.). Most typically, neoepitopes will be calculated to have a length of between 2-50 amino acids, more typically between 5-30 amino acids, and most typically between 9-15 amino acids, with a changed amino acid preferably centrally located or otherwise situated in a manner that ensures or improves its binding to MHC. For example, where the epitope is to be presented by the MHC-I complex, a typical neoepitope length will be about 8-11 amino acids, while the typical neoepitope length for presentation via MHC-II complex will have a length of about 13-17 amino acids. As will be readily appreciated, since the position of the changed amino acid in the neoepitope may be other than central, the actual peptide sequence and with that actual topology of the neoepitope may vary considerably.

Of course, it should be appreciated that the identification or discovery of neoepitopes may start with a variety of biological materials, including fresh biopsies, frozen or otherwise preserved tissue or cell samples, circulating tumor cells, exosomes, various body fluids (and especially blood), etc. Therefore, suitable methods of omics analysis include nucleic acid sequencing, and particularly NGS methods operating on DNA (e.g., Illumina sequencing, ion torrent sequencing, 454 pyrosequencing, nanopore sequencing, etc.), RNA sequencing (e.g., RNAseq, reverse transcription based sequencing, etc.), and protein sequencing or mass spectroscopy based sequencing (e.g., SRM, MRM, CRM, etc.).

As such, and particularly for nucleic acid based sequencing, it should be particularly recognized that high-throughput genome sequencing of a tumor tissue will allow for rapid identification of neoepitopes. However, it must be appreciated that where the so obtained sequence information is compared against a standard reference, the normally occurring inter-patient variation (e.g., due to SNPs, short indels, different number of repeats, etc.) as well as heterozygosity will result in a relatively large number of potential false positive neoepitopes. Notably, such inaccuracies can be eliminated where a tumor sample of a patient is compared against a matched normal (i.e., non-tumor) sample of the same patient.

In one especially preferred aspect of the inventive subject matter, DNA analysis is performed by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAMBAM format, SAMBAM format, FASTQ format, or FASTA format. However, it is especially preferred that the data sets are provided in BAMBAM format or as BAMBAM diff objects (see e.g., US2012/0059670A1 and US2012/0066001A1). Moreover, it should be noted that the data sets are reflective of a tumor and a matched normal sample of the same patient to so obtain patient and tumor specific information. Thus, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

Likewise, the computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Viewed from a different perspective, a patient- and cancer-specific in silico collection of sequences can be established that have a predetermined length of between 5 and 25 amino acids and include at least one changed amino acid. Such collection will typically include for each changed amino acid at least two, at least three, at least four, at least five, or at least six members in which the position of the changed amino acid is not identical. Such collection can then be used for further filtering (e.g., by sub-cellular location, transcription/expression level, MHC-I and/or II affinity, etc.) as is described in more detail below.

Depending on the type and stage of the cancer, it should be noted that not all of the identified neoepitopes will necessarily lead to a therapeutically equally effective reaction in a patient. Indeed, it is well known in the art that only a fraction of neoepitopes will generate an immune response. To increase likelihood of a therapeutically desirable response, neoepitopes can be further filtered. Of course, it should be appreciated that downstream analysis need not take into account silent mutations for the purpose of the methods presented herein. However, preferred mutation analyses will provide in addition to the type of mutation (e.g., deletion, insertion, transversion, transition, translocation) also information of the impact of the mutation (e.g., non-sense, missense, etc.) and may as such serve as a first content filter through which silent mutations are eliminated. For example, neoepitopes can be selected for further consideration where the mutation is a frame-shift, non-sense, and/or missense mutation.

In a further filtering approach, neoepitopes may also be subject to detailed analysis for sub-cellular location parameters. For example, neoepitope sequences may be selected for further consideration if the neoepitopes are identified as having a membrane associated location (e.g., are located at the outside of a cell membrane of a cell) and/or if an in silico structural calculation confirms that the neoepitope is likely to be solvent exposed, or presents a structurally stable epitope (e.g., J Exp Med 2014), etc.

With respect to filtering neoepitopes, it is generally contemplated that neoepitopes are especially suitable for use herein where omics (or other) analysis reveals that the neoepitope is actually expressed. Identification of expression and expression level of a neoepitope can be performed in all manners known in the art and preferred methods include quantitative RNA (hnRNA or mRNA) analysis and/or quantitative proteomics analysis. Most typically, the threshold level for inclusion of neoepitopes will be an expression level of at least 20%, at least 30%, at least 40%, or at least 50% of expression level of the corresponding matched normal sequence, thus ensuring that the (neo)epitope is at least potentially ‘visible’ to the immune system. Consequently, it is generally preferred that the omics analysis also includes an analysis of gene expression (transcriptomic analysis) to so help identify the level of expression for the gene with a mutation.

There are numerous methods of transcriptomic analysis known in the art, and all of the known methods are deemed suitable for use herein. For example, preferred materials include mRNA and primary transcripts (hnRNA), and RNA sequence information may be obtained from reverse transcribed polyA+-RNA, which is in turn obtained from a tumor sample and a matched normal (healthy) sample of the same patient. Likewise, it should be noted that while polyA+-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Similarly, proteomics analysis can be performed in numerous manners to ascertain actual translation of the RNA of the neoepitope, and all known manners of proteomics analysis are contemplated herein. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)). Consequently, it should be appreciated that the above methods will provide patient and tumor specific neoepitopes, which may be further filtered by sub-cellular location of the protein containing the neoepitope (e.g., membrane location), the expression strength (e.g., overexpressed as compared to matched normal of the same patient), etc.

In yet another aspect of filtering, the neoepitopes may be compared against a database that contains known human sequences (e.g., of the patient or a collection of patients) to so avoid use of a human-identical sequence. Moreover, filtering may also include removal of neoepitope sequences that are due to SNPs in the patient where the SNPs are present in both the tumor and the matched normal sequence. For example, dbSNP (The Single Nucleotide Polymorphism Database) is a free public archive for genetic variation within and across different species developed and hosted by the National Center for Biotechnology Information (NCBI) in collaboration with the National Human Genome Research Institute (NHGRI). Although the name of the database implies a collection of one class of polymorphisms only (single nucleotide polymorphisms (SNPs)), it in fact contains a relatively wide range of molecular variation: (1) SNPs, (2) short deletion and insertion polymorphisms (indels/DIPs), (3) microsatellite markers or short tandem repeats (STRs), (4) multinucleotide polymorphisms (MNPs), (5) heterozygous sequences, and (6) named variants. The dbSNP accepts apparently neutral polymorphisms, polymorphisms corresponding to known phenotypes, and regions of no variation. Using such database and other filtering options as described above, the patient and tumor specific neoepitopes may be filtered to remove those known sequences, yielding a sequence set with a plurality of neoepitope sequences having substantially reduced false positives.

Nevertheless, despite filtering, it should be recognized that not all neoepitopes will be visible to the immune system as the neoepitopes also need to be presented on the MHC complex of the patient. Indeed, only a fraction of the neoepitopes will have sufficient affinity for presentation, and the large diversity of MHC complexes will preclude use of most, if not all, common neoepitopes. Consequently, in the context of immune therapy it should thus be readily apparent that neoepitopes will be more likely effective where the neoepitopes are bound to and presented by the MHC complexes. Viewed from another perspective, treatment success with checkpoint inhibitors requires multiple neoepitopes to be presented via the MHC complex in which the neoepitope must have a minimum affinity to the patient's HLA-type. Consequently, it should be appreciated that effective binding and presentation is a combined function of the sequence of the neoepitope and the particular HLA-type of a patient. Most typically, the HLA-type determination includes at least three MHC-I sub-types (e.g., HLA-A, HLA-B, HLA-C) and at least three MHC-II sub-types (e.g., HLA-DP, HLA-DQ, HLA-DR), preferably with each subtype being determined to at least 2-digit depth or at least 4-digit depth. However, greater depth (e.g., 6 digit, 8 digit) is also contemplated herein. HLA determination can be performed using various methods in wet-chemistry that are well known in the art, and all of these methods are deemed suitable for use herein. Alternatively, the HLA-type can also be predicted from the patient omics data in silico using a reference sequence containing most or all of the known and/or common HLA-types as is shown in PCT/US16/48768.

Once the HLA-type of the patient is ascertained (using known chemistry or in silico determination), a structural solution for the HLA-type is calculated or obtained from a database, which is then used in a docking model in silico to determine binding affinity of the (typically filtered) neoepitope to the HLA structural solution. Suitable systems for determination of binding affinities include the NetMHC platform (see e.g., Nucleic Acids Res. 2008 Jul. 1; 36(Web Server issue): W509-W512.). Neoepitopes with high affinity (e.g., less than 100 nM, less than 75 nM, less than 50 nM) for a previously determined HLA-type are then selected for therapy creation, along with the knowledge of the MHC-I/II subtype.

More specifically, once patient and tumor specific neoepitopes and HLA-type are identified, computational analysis can be performed by docking neoepitopes to the HLA and determining best binders (e.g., lowest KD, for example, less than 500 nM, or less than 250 nM, or less than 150 nM, or less than 50 nM), for example, using NetMHC. Of course, it should be appreciated that matching of the patient's HLA-type to the patient- and cancer-specific neoepitope can be done using systems other than NetMHC, and suitable systems include NetMHC II, NetMHCpan, IEDB Analysis Resource (URL immuneepitope.org), RankPep, PREDEP, SVMHC, Epipredict, HLABinding, and others (see e.g., J Immunol Methods 2011;374:1-4).

In calculating the highest affinity, it should be noted that the collection of neoepitope sequences in which the position of the altered amino acid is moved (supra) can be used. Alternatively, or additionally, modifications to the neoepitopes may be implemented by adding N- and/or C-terminal modifications to further increase binding of the expressed neoepitope to the patient's HLA-type. Thus, neoepitopes may be native as identified or further modified to better match a particular HLA-type. Moreover, where desired, binding of corresponding wildtype sequences (i.e., neoepitope sequence without amino acid change) can be calculated to ensure high differential affinities. For example, especially preferred high differential affinities in MHC binding between the neoepitope and its corresponding wildtype sequence are at least 2-fold, at least 5-fold, at least 10-fold, at least 100-fold, at least 500-fold, at least 1000-fold, etc.).

It should be recognized that such approach will not only identify specific neoepitopes that are genuine to the patient and tumor, but also those neoepitopes that are most likely to be presented on a cell and as such most likely to elicit an immune response with therapeutic effect. Of course, it should also be appreciated that thusly identified HLA-matched neoepitopes can be biochemically validated in vitro prior to inclusion of the nucleic acid encoding the epitope as payload into the virus as is further discussed below. Additionally, it should be appreciated that HLA matching of neoepitopes will allow for intentional targeting of a neoepitope sequence toward MHC-I and/or MHC-II presentation, which in turn will allow for control over the immune response with respect to activation of CD8+ and CD4+ T cells (which will affect at least to some degree the balance between humoral and cellular immune response). For example, where a particular neoepitope will not elicit an effective immune response via presentation by the MHC-I pathway, the same neoepitope can be alternatively (or additionally) targeted for presentation by the MHC-II pathway. Still further, it is contemplated that expression of the neoepitopes may be exclusively or predominantly (e.g., at least 50%, or 60%, or 70%, or 80% of all neoepitopes) directed towards one presentation system. For example, where a more cellular immune response (e.g., ADCC by T cells response) is desired, presentation may be driven towards MHC-I presentation. On the other hand, where a more humoral response is desired (e.g., antibody/complement response), presentation may be driven towards MHC-II presentation.

With respect to routing the so identified and expressed neoepitopes to the desired MHC-system, it should be appreciated that the MHC-I presented peptides will typically arise from the cytoplasm via proteasome processing and delivery through the endoplasmatic reticulum. Thus, expression of the epitopes intended for MHC-I presentation will generally be directed to the cytoplasm as is further discussed in more detail below. On the other hand, MHC-II presented peptides will typically arise from the endosomal and lysosomal compartment via degradation and processing by acidic proteases (e.g., legumain, cathepsin L and cathepsin S) prior to delivery to the cell membrane. Thus, expression of the epitopes intended for MHC-II presentation will generally be directed to the endosomal and lysosomal compartment as is also discussed in more detail below.

In preferred aspects, signal peptides may be used for trafficking to the endosomal and lysosomal compartment, or for retention in the cytoplasmic space. For example, where the peptide is to be exported to the endosomal and lysosomal compartment selected targeting pre-sequences and internal targeting peptides can be employed. The pre-sequences of the targeting peptide are preferably added to the N-terminus and will typically comprise between 6-136 basic and hydrophobic amino acids. In case of peroxisomal targeting, the targeting sequence may be at the C-terminus. Other signals (e.g., signal patches) may be used and include sequence elements that are separate in the peptide sequence and become functional upon proper peptide folding. In addition, protein modifications like glycosylations can induce targeting. Among other suitable targeting signals, the inventors contemplate peroxisome targeting signal 1 (PTS1), a C-terminal tripeptide, and peroxisome targeting signal 2 (PTS2), which is a nonapeptide located near the N-terminus. In addition, sorting of proteins to endosomes and lysosomes may also be mediated by signals within the cytosolic domains of the proteins, typically comprising short, linear sequences. Some signals are referred to as tyrosine-based sorting signals and conform to the NPXY or YXXØ consensus motifs. Other signals known as dileucine-based signals fit [DE]XXXL[LI] or DXXLL consensus motifs. All of these signals are recognized by components of protein coats peripherally associated with the cytosolic face of membranes. YXXØ and [DE]XXXL[LI] signals are recognized with characteristic fine specificity by the adaptor protein (AP) complexes AP-1, AP-2, AP-3, and AP-4, whereas DXXLL signals are recognized by another family of adaptors known as GGAs. Also FYVE domain can be added, which has been associated with vacuolar protein sorting and endosome function. In still further aspects, endosomal compartments can also be targeted using human CD1 tail sequences (see e.g., Immunology, 122, 522-531).

Trafficking to or retention in the cytosolic compartment may not necessarily require one or more specific sequence elements. However, in at least some aspects, N- or C-terminal cytoplasmic retention signals may be added, including a membrane-anchored protein or a membrane anchor domain of a membrane-anchored protein. For example, membrane-anchored proteins include SNAP-25, syntaxin, synaptoprevin, synaptotagmin, vesicle associated membrane proteins (VAMPs), synaptic vesicle glycoproteins (SV2), high affinity choline transporters, Neurexins, voltage-gated calcium channels, acetylcholinesterase, and NOTCH.

In addition to specific targeting of proteins to the MHC-I and/or MHC-II system, it should be appreciated that the processing and presentation may be further enhanced by one or more signals that help accelerate protein turnover within the cell, for example, by the suitable choice of the N-terminal amino acid of the recombinant antigen or neoepitope. For example, to increase turnover, it is contemplated that the N-terminal amino acid may be a destabilizing amino acid. Thus, suitable N-terminal amino acids especially include Arg, His, Ile, Leu, Lys, Phe, Trp, and Tyr, and to some degree also Asn Asp, Gln, and Glu. Such amino acids may be added to peptides that are targeted to the MHC-I and/or MHC-II presentation pathways. In addition, it should be appreciated that protein turnover may also be enhanced using ubiquitin at the protein terminus, preferably coupled to by a non-cleavable linker.

Consequently, addressing the peptides to the appropriate compartments with suitable signal sequences, and optionally modifying the peptides with destabilizing N-terminal amino acids, will help increase antigen processing and presentation, which will also ultimately lead to antigen cascading and epitope spread.

Of course, it should be recognized that more than one tumor related antigen may be encoded in a recombinant nucleic acid, and that the arrangement of multiple antigens may vary considerably. For example, contemplated transcription or translation units may have concatemeric arrangement of multiple epitopes, typically separated by short linkers (e.g., flexible linkers having between 4 and 20 amino acids), which may further include protease cleavage sites. Especially suitable linker sequences will be designed such that the linker as well as the fusion portion between the linker and the tumor related antigen will not form a protein sequence that is normally present in the patient. Such concatemers may have between 1 and 20 neoepitopes (typically limited by size of recombinant nucleic acid that can be delivered via a virus), and it should be noted that the concatemers may be identical for delivery to the MHC-I and MHC-II complex, or different. Therefore, it should be appreciated that various peptides can be routed to specific cellular compartments to so achieve preferential or even specific presentation via MHC-I and/or MHC-II. Viewed from another perspective, it should be recognized that tumor associated antigens and neoepitopes may be presented via both presentation pathways, or selectively to one or another pathway at the same time or in subsequent rounds of treatment.

Additionally, it is preferred that the viral recombinant nucleic acid also encodes at least one, more typically at least two, even more typically at least three, and most typically at least four co-stimulatory molecules to enhance the interaction between the infected dendritic cells and T cells. For example, suitable co-stimulatory molecules include ICAM-1 (CD54), ICOS-L, and LFA-3 (CD58), especially in combination with B7.1 (CD80) and/or B7.2 (CD86). Further contemplated co-stimulatory molecules include 4-1BBL, CD30L, CD40, CD40L, CD48, CD70, CD112, CD155, GITRL, OX40L, and TL1A. Moreover, it should be appreciated that expression of the co-stimulatory molecules will preferably be coordinated such that the antigens and/or neoepitopes are presented along with the expression of one or more co-stimulatory molecules. Thus, it is typically contemplated that the co-stimulatory molecules are produced from a single transcript using an internal ribosome entry site or 2A sequence, or from multiple transcripts.

Additional examples of stimulatory factors to enhance immunogenicity include the following: (a) CD27 and CD70: The positive agonist CD27 and/or an biologic (e.g., antibody, ligand, etc.) that mimics CD27 interaction with CD70 on the T cell; (b) CD40 and CD40L: The positive agonist CD40 and/or biologic that mimics CD40 interaction with CD40L on the T cell; (c) OX40L and OX40: The positive agonist OX40L and/or biologic that mimics OX40L interactions with OX40 on the T cell; (d) GITRL and GITR: The positive agonist GITRL and/or biologic that mimics GITRL interactions with GITR on the T cell; (e) IL-2 and CD122: The positive agonist IL-2 and/or biologic that mimics IL-2 interactions with the IL-2 receptor on the T cell (e.g., CD122, etc.); (f) CD137 or an antibody the mimics CD137 activity with respect to the T cell; and (g) ICOSL and ICOS: The positive agonist ICOSL and/or biologic that mimics ICOSL interactions with the ICOS on the T cell.

Additionally, it should be recognized that expression of any co-stimulatory molecule can be paired with expression of any other protein that interferes with checkpoint inhibition. For example, the expression of co-stimulatory protein CD28 may be paired with expression of an inhibitor of CTLA-4. The applicants further contemplate that additional stimulatory or inhibitory factors can be influenced via the payload of the virus. The viruses can include payloads that can be tailored to mimic the natural immune responses. For example, a first virus having an agonist (e.g., simulation of CD28) which aids in stimulating T cells can be administered to the patient. A second virus having an antagonist (e.g., inhibitor of CTLA-4) can be administered to the patient at a later time, which prevents an inhibitory response. It is also contemplated that the ordering of the delivery can be switched. Further, a single virus can be constructed support both the stimulatory and the inhibitory factors. Alternatively, co-stimulatory molecules may be co-expressed with the tumor related antigens, while checkpoint inhibitors may be (subcutaneously) injected.

Therefore, it is also contemplated that the recombinant virus will further include a sequence portion that encodes one or more peptide ligands that bind to a checkpoint receptor. Most typically, binding will inhibit or at least reduce signaling via the receptor, and particularly contemplated receptors include CTLA-4 (especially for CD8+ cells) and PD-1 (especially for CD4+ cells). For example, peptide binders can include antibody fragments and especially scFv, but also small molecule peptide ligands that specifically bind to the receptors. Once more, it should be appreciated that expression of the peptide molecules will preferably be coordinated such that the antigens and/or neoepitopes are presented along with one or more peptide molecules. Thus, it is typically contemplated that the peptide molecules are produced from a single transcript using an internal ribosome entry site or 2A sequence, or from multiple transcripts.

Further examples of inhibitory factors that can be enhanced via suitably constructed viruses are considered to include the following: (a) Naturally occurring or engineered ligands that inhibit CD276/B7-H3 inhibition of T cell activation; (b) Naturally occurring or engineered ligands that inhibit B7-H4/VTCN1 inhibition of T cell activation; (c) Naturally occurring or engineered ligands that inhibit CD272/HVEM inhibition of T cell activation; (d) Naturally occurring or engineered ligands (e.g., MHC-II, etc.) that inhibit LAG3 inhibition of T cell activation; (e) Naturally occurring or engineered ligands (e.g., PD-L1) that inhibit PD-1 inhibition of T cell activation; (f) Naturally occurring or engineered ligands (e.g., biologic, soluble CD28, etc.) that inhibit CTLA-4 inhibition of T cell activation; (g) Naturally occurring or engineered ligands (e.g., galectin-9, biologic, antibody, etc.) that inhibit TIM-3 inhibition of T cell activation; (h) Naturally occurring or engineered ligands (e.g., antibody, etc.) that inhibit VISTA inhibition of T cell activation; and (i) Naturally occurring or engineered ligands (e.g., antibody, biologic etc.) that inhibit MIC inhibition of NK cells.

Most typically, expression of the recombinant genes is driven from constitutively active regulatory sequences. However, in other aspects of the inventive subject matter, the regulatory sequences may be inducible, preferably in a selective manner using one or more regulatory signals endogenous to the cancerous tissue or synthetic inducers. For example, inducible expression may be performed using synthetic inducers or naturally occurring inducers in conjunction with appropriate response elements. In most cases, it is further preferred that the transcript will includes an IRES (internal ribosome entry site) or a 2A sequence (cleavable 2A-like peptide sequence) to again allow for coordinated expression of the tumor related antigens, co-stimulatory molecules, and/or checkpoint inhibitors.

Consequently, it should be appreciated that using contemplated systems and methods, immune therapy may be performed by expression of one or more tumor related antigens and co-stimulatory molecules in antigen presenting cells and especially dendritic cells, which is further performed in the presence of inhibitors of checkpoint inhibition (that may equally be expressed in the antigen presenting cell. Such coordinated event, particularly when directed towards specific MHC presentation is believed to produce an enhanced adaptive immune response that may be further complemented by administration of cellular components, and especially NK cells.

As will be readily appreciated, the tumor related antigens, co-stimulatory molecules, and/or checkpoint inhibitors will be encoded on a recombinant nucleic acids that may be administered as DNA vaccine or as RNA, but it is generally preferred that the recombinant nucleic acid is part of a viral genome. The so genetically modified virus can then be used as is well known in gene therapy. Thus, with respect to recombinant viruses it is contemplated that all known manners of making recombinant viruses are deemed suitable for use herein, however, especially preferred viruses are those already established in therapy, including adenoviruses, adeno-associated viruses, alphaviruses, herpes viruses, lentiviruses, etc. Among other appropriate choices, adenoviruses are particularly preferred.

Moreover, it is further generally preferred that the virus is a replication deficient and non-immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol. 1998 Feb.; 72(2): 926-933). As noted before, the desired nucleic acid sequences (for expression from virus infected cells) are under the control of appropriate regulatory elements well known in the art. In view of the above, it should therefore be appreciated that compositions and methods presented are not only suitable for directing virally expressed antigens specifically to one or another (or both) MHC systems, but will also provide increased stimulatory effect on the CD8+ and/or CD4+ cells via inclusion of various co-stimulatory molecules (e.g., ICAM-1 (CD54), ICOS-L, LFA-3 (CD58), and at least one of B7.1 (CD80) and B7.2 (CD86)), and via secretion or membrane bound presentation of checkpoint inhibitors.

So produced recombinant viruses may then be individually or in combination used as a therapeutic vaccine in a pharmaceutical composition, typically formulated as a sterile injectable composition with a virus titer of between 10⁴-10¹¹ virus particles per dosage unit. However, alternative formulations are also deemed suitable for use herein, and all known routes and modes of administration are contemplated herein. As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). Most preferably, the recombinant virus is administered via subcutaneous or subdermal injection. However, in other contemplated aspects, administration may also be intravenous injection. Alternatively, or additionally, antigen presenting cells may be isolated or grown from cells of the patient, infected in vitro, and then transfused to the patient.

In addition, it is contemplated that prophylactic or therapeutic administration of the recombinant virus may be accompanied by co-administration with one or more checkpoint inhibitors, especially where the recombinant virus does not include nucleic acid sequences encoding peptides that target the checkpoint receptors. For example, especially preferred check point inhibitors include currently available inhibitors (e.g., pembrolizumab, nivolumab, ipilimumab) that are (most preferably) administered subcutaneously at or near the site of the subcutaneous administration of the viral vector.

Consequently, as the recombinant virus is delivered to the dendritic and other antigen presenting cells in the dermal layers and presented via MHC-I and/or MHC-II pathways, it should be recognized that processing through the immune system will result in stimulation of both CD8+ and CD4+ cells, which will lead to formation of trained B-cells for formation of IgG₁, T cells, as well as trained NK cells and the corresponding memory cells. In addition, it should be noted that the IgG₁ molecules will also enable tumor specific action by NK cells.

Therefore, it is contemplated that treatment will preferably also include transfusion of autologous or heterologous NK cells to the patient, and particularly NK cells that are genetically modified to exhibit less inhibition. For example, the genetically modified NK cell may be a NK-92 derivative that is modified to have a reduced or abolished expression of at least one killer cell immunoglobulin-like receptor (KIR), which will render such cells constitutively activated. Of course, it should be noted that one or more KIRs may be deleted or that their expression may be suppressed (e.g., via miRNA, siRNA, etc.), including KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest as aNK cells ('activated natural killer cells). Such cells may then be further modified to express the co-stimulatory molecules as further discussed below. In addition, contemplated NK cells suitable for use herein also include those that have abolished or silenced expression of NKG2A, which is an activating signal to Tregs and MDSCs.

In another preferred aspect of the inventive subject matter, the genetically engineered NK cell may also be an NK-92 derivative that is modified to express the high-affinity Fcγ receptor (CD16). Sequences for high-affinity variants of the Fcγ receptor are well known in the art, and all manners of generating and expression are deemed suitable for use herein. Expression of such receptor is believed to allow specific targeting of tumor cells using antibodies produced by the patient in response to the treatment contemplated herein, or supplied as therapeutic antibodies, where those antibodies are specific to a patient's tumor cells (e.g., neoepitopes), a particular tumor type (e.g., her2neu, PSA, PSMA, etc.), or antigens associated with cancer (e.g., CEA-CAM). Advantageously, such cells may be commercially obtained from NantKwest as haNK cells ('high-affinity natural killer cells) and may then be further modified (e.g., to express co-stimulatory molecules as discussed above).

Alternatively, the genetically engineered NK cell may also be genetically engineered to express a chimeric T cell receptor. In especially preferred aspects, the chimeric T cell receptor will have an scFv portion or other ectodomain with binding specificity against a tumor associated antigen, a tumor specific antigen, and/or a neoepitope. As before, such cells may be commercially obtained from NantKwest as taNK cells (‘target-activated natural killer cells’) and further modified as desired. Where the cells have a chimeric T cell receptor engineered to have affinity towards a cancer associated antigen or neoepitope, it is contemplated that all known cancer associated antigens and neoepitopes are considered appropriate for use. For example, tumor associated antigens include CEA, MUC-1, CYPB1, PSA, Her-2, PSA, brachyury, etc.

Moreover, it should be noted that the compositions and methods contemplated herein also include cell based treatments with cells other than (or in addition to) NK cells. For example, suitable cell based treatments include T cell based treatments. Among other options, it is contemplated that one or more features associated with T cells (e.g., CD4+ T cells, CD8+ T cells, etc.) can be detected. More specifically, the GPS Cancer tests can provide specific neoepitopes (e.g., 8-mers to 12-mers for MHC I, 12-mers to 25-mers for MHC II, etc.) that can be used for the identification of neoepitope reactive T cells bearing a specific T cell receptor against the neoepitopes/MHC protein complexes. Thus, the method can include harvesting the neoepitope reactive T cells. The harvested T cells can be grown or expanded ex vivo in preparation for reintroduction to the patient. Alternatively, the T cell receptor genes in the harvested T cells can be isolated and transferred into viruses, or other adoptive cell therapies systems (e.g., CAR-T, CAR-TANK, etc.). Beyond neoepitopes, the GPS Cancer test can also provide one or more tumor associated antigens (TAAs). Therefore, one can also harvest T cells that have receptors that are sensitive to the TAAs identified from the test. These can also be grown or cultured ex vivo and used in a similar therapeutic manner as discussed above. The T cells can be identified by producing synthetic versions of the peptides and bind them with commercially produced MHC or MHC-like proteins, then using these ex vivo complexes to bind to the target T cells. One should appreciated that the harvested T cells can included T cells that have been activated by the patient's immune response to the disease, exhausted T cells, or other T cells that are responsive to the discussed features.

Exhausted T cells can be reactivated through several different routes. One route includes using exogenously adding cytokines (e.g., IL-2, IL-12, IL-15, etc.) to the harvested exhausted T cells to reinvigorate the cells. The reinvigorated T cells can then be reintroduced back to the patient, possibly along with a checkpoint inhibitors (e.g., ipilimumab, etc.). Another route is to prevent exhaustion through blockading checkpoint inhibition, which can be achieved through administering a tailored virus having the target neoepitopes and with an appropriate inhibitor (e.g., LAG3, etc.).

The applicants have further appreciated that the patient's bulk white blood cells (WBCs) can be cultured with the discovered peptides (e.g., TAA, neoepitopes, etc.) from the GPS Cancer tests. Such an approach is expected to cause production of desired MHC/neoepitope complexes by the antigen presenting cells in the bulk WBCs. Thus, the patient's macrophages, dendritic cells, and B-Cells provide instruction to the NK cells and T cells so that they take on the desired properties to target the diseased tissue.

Yet another interesting consideration related to the impact the gut biome has in a patient's immune response. Contemplated inventive subject matter also includes methods of identifying micro-biome produced epitopes, which are predicted to elicit a regulatory or immunosuppressive immune response. The set of identified epitopes can be removed from the set of neoepitopes discovered via GPS Cancer testing. It is thought that neoepitopes that are similar to the epitopes from the micro-biome would be less useful in targeting the disease tissue because the patient's body would already likely be tolerant to such similar peptides.

In view that the gut biome can influence the patient's immune response to the disease, the applicants further contemplate methods of treating a patient by administering antibiotics to the patient where the antibiotics target the gut micro-biome. For example, antibiotics can be given the patient to inhibit or suppress elements of the micro-biome that elicit inhibitory T cells (e.g., up-regulate Th2, Th17, and regulatory T cells) concurrent to introduction to the immunotherapy as discussed above. In other embodiments, the patient can be prescribed a diet that inhibits or suppresses the elements of the micro-biome.

Yet another consideration is that the applicants have pioneered comprehensive “omics” testing as a single test referenced as a GPS Cancer™ test or companion diagnostic. This single test provides numerous insights regarding the state of the patient's diseased tissue including the following types of information: whole genome sequences, RNA, RNAseq, proteomics, expression levels, and neoepitopes, among others. It should be appreciated that these results are patient-specific as well as disease-specific. Further, these results provide patient-specific and disease-specific guidance on a vast array of therapies targeting the disease. For example, the results can influence one or more of the following therapies to create a highly personalized treatment: chemotherapy, monoclonal antibody therapy, antibody therapy, small molecule therapy, immunotherapy, therapies directed to tumor associated antigens, or any combination of therapies. More specifically, a genomic sequence could inform which type of chemotherapy might be most relevant, while the neoepitopes inform construction of one or more viruses that, when administered to the patient, augment the patient's immune response toward the disease as discussed previously. In some embodiments, the single GPS Cancer test can be conducted repeatedly over time. The results of each test can then be brought to bear on modifying personalized therapy to better suit the patient's disease.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Finally, and unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a patient having a tumor, comprising:
subcutaneously administering a recombinant virus comprising a nucleic acid that encodes
(a) at least one tumor-related epitope of the tumor of the patient;
(b) at least one co-stimulatory molecule; and
(c) a peptide that binds to a checkpoint receptor;
wherein the nucleic acid further includes a trafficking signal to direct a peptide product encoded by the nucleic acid to the cytoplasm, the endosomal compartment, or the lysosomal compartment; and
administering NK cells to the patient.

2. The method of claim 1 wherein the recombinant virus is an adenovirus, optionally with a deleted or non-functional E2b gene.

3-13. (canceled)

14. The method of claim 1 wherein the tumor-related epitope is an HLA-matched tumor-related epitope, or wherein the tumor-related epitope is a cancer associated epitope, a cancer-specific epitope, or a patient- and tumor-specific neoepitope.

15. (canceled)

16. The method of claim 1 wherein the co-stimulatory molecule is B7.1 (CD80), B7.2 (CD86), ICAM-1 (CD54), ICOS-L, LFA-3 (CD58), 4-1BBL, CD30L, CD40,CD40L, CD48, CD70, CD112, CD155, GITRL, OX40L, or TL1A, or wherein the peptide that binds to the checkpoint receptor binds to CTLA-4 (CD152) or PD-1 (CD 279).

17. (canceled)

18. The method of claim 1 wherein the trafficking signal directs the peptide product to the cytoplasm.

19. The method of claim 1 wherein the trafficking signal directs the peptide product to the endosomal compartment or to the lysosomal compartment.

20. (canceled)

21. The method of claim 1 wherein the nucleic acid has a first trafficking signal that directs a first peptide product to the cytoplasm and a second trafficking signal that directs a second peptide product to the endosomal or lysosomal compartment.

22. The method of claim 1 wherein the peptide product further comprises a sequence portion that enhances intracellular turnover of the peptide product.

23. The method of claim 1 wherein the NK cells are genetically modified NK cells that (1) have a reduced or abolished expression of at least one killer cell immunoglobulin-like receptor, (2) express a high-affinity Fcγ receptor, (3) express a chimeric T cell receptor, and/or (4) have a deletion in NKG2A.

24. The method of claim 1 wherein the step of administering NK cells is performed between one and 14 days after subcutaneously administering the recombinant virus.

25. A method of stimulating a CD8+ T cell response in a patient having a tumor, comprising:
subcutaneously administering a recombinant virus that comprises a nucleic acid that encodes
(a) at least one tumor-related epitope of the tumor of the patient, operably coupled to a trafficking signal that retains the at least one tumor-related epitope in the cytoplasm;
(b) a plurality of co-stimulatory molecules, at least one of which is B7.1 (CD80) or B7.2 (CD86);
(c) a peptide that binds to at least one of PD-1 and CTLA-4; and
administering NK cells to the patient.

26-31. (canceled)

32. The method of claim 25 wherein the tumor-related epitope further comprises a sequence portion that enhances intracellular turnover of the tumor-related epitope.

33. The method of claim 25 wherein the tumor-related epitope is an HLA-matched cancer associated epitope, an HLA-matched cancer-specific epitope, or an HLA-matched patient- and tumor-specific neoepitope.

34. The method of claim 25 wherein the plurality of co-stimulatory molecules further comprises at least one additional co-stimulatory molecule selected form the group consisting of ICAM-1 (CD54), ICOS-L, LFA-3 (CD58), 4-1BBL, CD30L, CD40, CD40L, CD48, CD70, CD112, CD155, GITRL, OX40L, and TL1A, or wherein the peptide that binds to at least one of PD-1 and CTLA-4 is a membrane bound antibody fragment.

35. (canceled)

36. The method of claim 25 wherein the NK cells are genetically modified NK cells that (1) have a reduced or abolished expression of at least one killer cell immunoglobulin-like receptor, (2) express a high-affinity Fcγ receptor, (3) express a chimeric T cell receptor, and/or (4) have a deletion in NKG2A.

37. A method of stimulating a CD4+ T cell response in a patient having a tumor, comprising:
subcutaneously administering a recombinant virus that comprises a nucleic acid that encodes
(a) at least one tumor-related epitope of the tumor of the patient, operably coupled to a trafficking signal that directs the at least one tumor-related epitope to the cytoplasm or the endosomal or lysosomal compartment;
(b) a plurality of co-stimulatory molecules, at least one of which is B7.1 (CD80) or B7.2 (CD86); and
(c) a peptide that binds to at least one of PD-1 and CTLA-4;
administering NK cells to the patient.

38-43. (canceled)

44. The method of claim 37 wherein the tumor-related epitope further comprises a sequence portion that enhances intracellular turnover of the tumor-related epitope.

45. The method of claim 37 wherein the tumor-related epitope is an HLA-matched cancer associated epitope, an HLA-matched cancer-specific epitope, or an HLA-matched patient- and tumor-specific neoepitope.

46. The method of claim 37 wherein the plurality of co-stimulatory molecules further comprises at least one additional co-stimulatory molecule selected form the group consisting of ICAM-1 (CD54), ICOS-L, LFA-3 (CD58), 4-1BBL, CD30L, CD40, CD40L, CD48, CD70, CD112, CD155, GITRL, OX40L, and TL1A, or wherein the peptide that binds to at least one of PD-1 and CTLA-4 is a membrane bound antibody fragment.

47. (canceled)

48. The method of claim 37 wherein the NK cells are genetically modified NK cells that (1) have a reduced or abolished expression of at least one killer cell immunoglobulin-like receptor, (2) express a high-affinity Fcγ receptor, (3) express a chimeric T cell receptor, and/or (4) have a deletion in NKG2A.

49-72. (canceled)
